(19) United States
(12) Patent Application Publication
(10) Pub. No.: US 2010/0218266 A1
(43) Pub. Date: Aug. 26, 2010

(54) METHODS OF IDENTIFYING AND USING SNAIL1 INHIBITORY COMPOUNDS IN CHONDRODYSPLASIA TREATMENT AND PREPARATION OF PHARMACEUTICAL COMPOSITIONS

(76) Inventors: Maria Angela Nieto Toledano, San Juan De Alicante (ES); Cristina Alvarez De Frutos, San Juan De Alicante (ES); Sonia Vega, San Juan De Alicante (ES)

Correspondence Address: IP Patent Docketing;K&L GATES LLP, 599 Lexington Avenue, 33rd Floor, New York, NY, 10022-6030, US

(21) Appl. No.: 12/530,347

(22) PCT Filed: Mar. 7, 2008
(86) PCT No.: PCT/ES2008/070042

§ 371 Date: Feb. 12, 2010

(30) Foreign Application Priority Data
Mar. 8, 2007 (ES) 200700619

(51) Int. Cl. A61K 31/713; C07H 21/02; C12Q 1/02; G01N 33/53; A01K 67/027; A61P 43/00

(52) U.S. Cl. 800/18; 536/24.5; 514/44 A; 435/29; 435/7.1

(57) ABSTRACT

Exemplary embodiments disclosed herein demonstrate that the Snail1 gene contributes to FGFR3 receptor signal transduction, which contributes to chondrodysplasias (achondroplasia (ACH), thanatophoric dysplasia (TD) and hypochondroplasia (HCH)). The exemplary embodiments identify Snail1 as a therapeutic and diagnostic target for chondrodysplasia, as well as the use of inhibitors thereof as drugs for the treatment of these diseases.

### CROSS-REFERENCES TO RELATED APPLICATIONS

This application claims the benefit under 35 U.S.C. §371 to PCT/ES2008/070042 filed Mar. 7, 2008, which claims the benefit of Patent Application 200700619 filed Mar. 8, 2007 in Spain. The entire disclosures of both applications are incorporated by reference herein.

### STATE OF THE ART

The skeleton is formed by cartilages and bones. In turn, cartilages are formed by chondrocytes, whereas bones are formed by osteoblasts and osteoclasts. Chondrocytes and osteoblasts have a mesenchymal origin, whereas osteoclasts arise from the haematopoietic lineage.

Chondrocytes play an essential role in the formation of most skeletal components. In addition to their role during osteogenesis, the chondrocytes located in the growth plate control the longitudinal growth of bones. They gradually become differentiated inside the mesenchymal condensations, from proliferative chondrocytes until they reach their final differentiation stage as hypertrophic chondrocytes. This cell population gradually becomes surrounded by a calcified extracellular matrix, which favors the invasion of blood vessels from the perichondrium. This is when perichondrium cells begin to differentiate and become osteoblasts, forming the mineralized structure called bone collar.

After vascular invasion, hypertrophic chondrocytes die by apoptosis and osteoblasts begin to deposit the bone extracellular matrix, which is primarily composed of type I collagen. Chondrocytes are restricted to the growth plate, where, jointly with osteoblasts, they direct longitudinal bone growth.

In the growth plate, members of the FGF family and the receptors thereof, primarily receptor 3 (FGFR3), regulate the proliferation and differentiation of chondrocytes, inhibiting bone growth. FGFR3 is expressed in proliferative chondrocytes. Gain-of-function mutations of Fgfr3 cause hypochondroplasia, achondroplasia, and thanatophoric dysplasia, the most severe variant of achondroplasia. Hypochondroplasia (HCH, OMIM 1460000) is the mildest form of this type of dwarfism and in 65% of cases is caused by mutation N540K in the tyrosine kinase 1 domain of the receptor. Achondroplasia (ACH, OMIM 100800), which in 98% of cases is caused by mutation 0380R in the transmembrane domain of the receptor, is the most common chondrodysplasia in humans. Thanatophoric dysplasia presents a lethal phenotype and two varieties have been described by radiological diagnosis: type I and type II (TD I and TD II, OMIM 187600 and 187601, respectively). There exist murine models that reproduce the pathologies with mutations in the corresponding positions. In all cases, the long-bone shortening phenotype is due to a disorganization and shortening of the proliferative chondrocyte columns and to delayed differentiation. Defects in the proliferative chondrocytes are due to the activation of Stat1, which is responsible for the induction of cell cycle inhibitor p21, and delayed differentiation is due to the activation of the MAPK signaling cascade, which causes a reduction in the area of hypertrophic chondrocytes, in both animal and human models. On the contrary, the de-activation of Fgfr3 in mice causes prolonged endochondral growth, resulting in a “long-bone” phenotype, which is accompanied by an expansion of the proliferative chondrocyte region in the growth plate. All these data give FGFR3 a significant role as a negative regulator of the proliferation of chondrocytes. This inhibition of proliferation by the FGF pathway is unique to chondrocytes and is mediated by transcription factor STAT1, which increases the expression of cell cycle inhibitor p21, the final agent responsible for the interruption of the proliferation induced by this signaling pathway. Its levels can be considered to be a reflection of the activation of the FGFR3-mediated signaling pathway in the growth plate.

When mouse Snail was cloned, it was observed that on day 12 in the embryonic development of the mouse, the predominant expression site of Snail was the pre-cartilage, including the pre-cartilages corresponding to the tail schlerotome, pre-vertebrae, ribs, limbs, and head. However, from day 14 in development, there is no expression of any of these sites in the pre-cartilage any more, except for the distal phalanges of the posterior extremities, which are the only sites where there is still pre-cartilage in the legs at this stage.

Snail1 has been recently described as a direct repressor of type II collagen, which is characteristic of proliferative chondrocytes, and disappears when the latter cease to proliferate and differentiate into hypertrophic chondrocytes, the cell population that expresses type X collagen. In a completely independent context, it was observed that the presence of Snail attenuated the proliferation of epithelial cells in culture and evolved with an increase in p21 levels and an increase in the phosphorylation of ERK1 and ERK2.

In summary, human chondrodysplasias (which evolve with a delay and irregularity in cartilage formation) and the murine models generated to study them have been associated with mutations that generate a greater FGFR3 activity, which causes delayed differentiation and interruption of the proliferation of pathological chondrocyte populations, thereby preventing the individual's correct bone development.

There are several therapeutic approaches to these human chondrodysplasias. The surgical approach is very invasive and of long duration. Treatment with growth hormones is not very effective in achondroplasia, aggravates the disproportion between the trunk and the extremities, and is very costly. The use of natiuretic peptide CNP inhibits the FGF-mediated MAPK activation in the growth plate, but does not salvage the defects in the proliferation of chondrocytes. Other strategies intend to decrease the receptor's tyrosine kinase activity with chemical agents or block binding of the ligand to FGFR3 with antibodies.

### BRIEF DESCRIPTION

Exemplary embodiments disclosed herein include a method for identifying a chondrodysplasia process based on the identification of the presence of Snail1 in a biological sample, which can comprise the following steps: a) identifying the presence of Snail1 in a biological sample of osseous origin, and b) comparing the presence of Snail1 observed in a) to its absence in a control sample, where its presence is indicative of the existence of chondrodysplasia.

Another exemplary embodiment is characterized in that the chondrodysplasia process is a disease with a dwarfism-type chondrodysplasia phenotype wherein the biological action of Snail1 is the cause of the disease and is accompanied by an anomalous activation of receptor FGFR3.

Another exemplary embodiment is characterized in that the disease belongs to the following group: achondroplasia (ACH), thanatophoric dysplasia (TD) or hypochondroplasia (HCH).

Another exemplary embodiment is characterized in that the identification of Snail1 refers to both the Snail1 gene and the protein that is the product of the expression thereof.

Another exemplary embodiment is characterized in that Snail1 is the mouse Snail1 gene or protein with the sequence SEQ ID NO: 1 or SEQ ID NO: 2, respectively.

Another exemplary embodiment is characterized in that Snail1 is the human Snail1 gene or protein with the sequence SEQ ID NO: 3 or SEQ ID NO: 4, respectively.

Another exemplary embodiment is characterized in that the identification of Snail1 of step (a) refers to the human form of Snail1, whether the identification is performed in the form of a gene transcript (mRNA) or the protein form of the gene, with sequence SEQ ID NO: 3 or SEQ ID NO 4.

Another exemplary embodiment is characterized in that the identification of Snail1 is performed using specific antibodies, either monoclonal or polyclonal, of the hSnail1 protein.

Another exemplary embodiment is characterized in that the identification of Snail1 is performed by means of in situ hybridization with a Snail1 precursor.

Another exemplary embodiment is characterized in that the identification of Snail1 is performed by means of reverse transcriptase polymerase chain reaction (RT-PCR) of a Snail1 precursor.

One exemplary embodiment includes a method for identifying a chondrodysplasia process in a mammal comprising: a) identifying the aberrant presence of Snail1 in a biological sample of osseous origin from the mammal by b) comparing the presence of Snail1 observed in a) to its absence in a control sample, where its presence is indicative of the existence of chondrodysplasia.

In another exemplary embodiment, the identifying comprises identification of Snail1 mRNA or protein expression.

In another exemplary embodiment, the identifying comprises identification of SEQ ID NO: 1, SEQ ID NO: 2, SEQ ID NO: 3 or SEQ ID NO: 4.

In another exemplary embodiment, the identifying is performed using monoclonal or polyclonal antibodies of the hSnail1 protein, in situ hybridization with a Snail1 precursor, or RT-PCR of a Snail1 precursor.

Exemplary embodiments disclosed herein include a method for identifying and evaluating the activity of inhibitory compounds of the Snail1 protein that can be useful in the treatment of chondrodysplasia which can comprise the following steps: a) placing a biological system with an expression of Snail1 that produces chondrodysplasia in contact with a candidate compound, with incubation under suitable conditions, b) determining a parameter that is indicative of the chondrodysplasia process, and c) identifying a compound inhibitory of Snail1 protein activity when a reduction of the chondrodysplasia parameter is observed.

One exemplary embodiment is characterized in that the biological system of step (a) is a non-human transgenic animal, where the expression of the Snail1 protein is inducible, in a constant or conditional manner, and the expression thereof causes chondrodysplasia.

Another exemplary embodiment is characterized in that the transgenic animal used is the transgSnail1-ER transgenic mouse.

In another exemplary embodiment, the inducible Snail1 expression is constant or conditional and causes chondrodysplasia.

In another exemplary embodiment, the non-human transgenic animal is the transgSnail1-ER transgenic mouse.

Exemplary embodiments disclosed herein include a biological system necessary for performing the method for identifying compounds, in one exemplary embodiment a transgenic animal, in one exemplary embodiment a mammal, and, in one exemplary embodiment, a non-human primate, where the expression of the Snail1 protein is inducible, in a constant or conditional manner, and where the expression thereof causes chondrodysplasia. A particular embodiment of the non-human mammalian animal is the transgenic mouse transgSnail1-ER described in Example 2.

One exemplary embodiment is characterized in that it is a non-human transgenic animal that may be induced to express the Snail1 protein, in a constant or conditional manner, and where the expression thereof causes chondrodysplasia.

Another exemplary embodiment is characterized in that the non-human transgenic animal is the transgSnail1-ER transgenic mouse.

Exemplary embodiments disclosed herein include the use of a compound or agent inhibitory of Snail1 protein activity in the preparation of a drug or pharmaceutical composition useful in the treatment of a chondrodysplasia process, in one exemplary embodiment for human or veterinary use.

Exemplary embodiments disclosed herein include the use of an inhibitory compound of Snail1 wherein the inhibitory compound can be a nucleic acid or polynucleotide that can prevent or reduce the expression of the gene that encodes the human Snail1 protein and which can include a nucleotide sequence selected from: a) an anti-sense nucleotide sequence specific to the gene or mRNA sequence of the Snail1 protein, b) a ribozyme specific to the mRNA of the Snail1 protein, c) an aptamer specific to the mRNA of the Snail1 protein, d) an interference RNA (sRNA or shRNA) specific to the mRNA of the Snail1 protein, and e) a microRNA (miRNA) specific to the Snail1 protein.

Exemplary embodiments disclosed herein include a pharmaceutical composition or a drug useful in the treatment of a chondrodysplasia process, hereinafter pharmaceutical composition, which can comprise use of a therapeutically effective quantity of an inhibitory compound or agent of the Snail1 protein, jointly with, optionally, one or more pharmaceutically acceptable adjuvants and/or carriers.

One exemplary pharmaceutical composition embodiment includes a pharmaceutical composition to be used in the treatment of a chondrodysplasia process, characterized in that it comprises a therapeutically effective quantity of an inhibitory compound or agent of the Snail1 protein, jointly with, optionally, one or more pharmaceutically acceptable adjuvants and/or carriers, where the inhibitory compound or agent is a nucleic acid or polynucleotide that prevents or reduces the expression of the gene that encodes the Snail1 protein and includes, at least, a nucleotide sequence selected from: a) an anti-sense nucleotide sequence specific to the gene or mRNA sequence of the Snail1 protein, b) a ribozyme specific to the mRNA, of the Snail1 protein, c) an aptamer specific to the mRNA of the Snail1 protein, d) an interference RNA (siRNA or shRNA) specific to the mRNA of the Snail1 protein, and e) a microRNA (miRNA) specific to the Snail1 protein.

Another exemplary pharmaceutical composition embodiment includes siRNA that binds to Snail mRNA fragment sequence SEQ ID NO: 17 or to another sequence that comprises the latter or to a shorter fragment thereof.

Another exemplary pharmaceutical composition embodiment includes siRNA composed of a pair of nucleotide sequences, or a mixture thereof, belonging to the following group: -I: SEQ ID NO 11 and the complementary thereof, SEQ ID NO 12, -II: SEQ ID NO 13 and the complementary thereof, SEQ ID NO 14, or -III: SEQ ID NO 15 and the complementary thereof, SEQ ID NO 16.

Another exemplary pharmaceutical composition embodiment includes one for use where the chondrodysplasia process is selected from the list that comprises achondroplasia (ACH), thanatophoric dysplasia (TD) and hypochondroplasia (HCH).

Another exemplary pharmaceutical composition embodiment includes a pharmaceutical composition useful in the treatment of a chondrodysplasia process comprising an inhibitory compound of the Snail1 protein.

In another exemplary embodiment, the pharmaceutical composition further comprises pharmaceutically acceptable adjuvants and/or carriers.

In another exemplary embodiment, the inhibitor compound is a nucleic acid or polynucleotide that prevents or reduces the expression of the gene that encodes the Snail1 protein.

In another exemplary embodiment, the inhibitory compound is one or more of: a) an anti-sense nucleotide sequence specific to the gene or mRNA sequence of the Snail1 protein, b) a ribozyme specific to the mRNA of the Snail1 protein, c) an aptamer specific to the mRNA of the Snail1 protein, d) siRNA or shRNA specific to the mRNA of the Snail1 protein, or e) a microRNA specific to the Snail1 protein.

In another exemplary embodiment, the inhibitory compound is siRNA that binds to SEQ ID NO: 17, a sequence comprising SEQ ID NO: 17 or a fragment of SEQ ID NO: 17.

In another exemplary embodiment, the siRNA comprises SEQ ID NO 11, SEQ ID NO 12, SEQ ID NO 13, SEQ ID NO 14, SEQ ID NO 15 and/or SEQ ID NO 16.

In another exemplary embodiment, the siRNA comprises a pair of nucleotide sequences including SEQ ID NO 11 and SEQ ID NO 12, SEQ ID NO 13 and SEQ ID NO 14 and SEQ ID NO 15 and SEQ ID NO 16.

Exemplary embodiments disclosed herein include the use of a pharmaceutical composition in a treatment method for a mammal, in one exemplary embodiment a human being, affected by a chondrodysplasia process which can consist in administering the therapeutic composition that can inhibit the chondrodysplasia process.

One exemplary embodiment includes a method of treating a mammal affected by a chondrodysplasia process comprising administering a therapeutically effective amount of a pharmaceutical composition disclosed herein. In another embodiment, the chondrodysplasia process is achondroplasia (ACH), thanatophoric dysplasia (TD) or hypochondroplasia (HCH). In another embodiment, the siRNA binds to SEQ ID NO:17, a sequence comprising SEQ ID NO: 17 or a fragment of SEQ ID NO: 17 and includes SEQ ID NO 11, SEQ ID NO 12, SEQ ID NO 13, SEQ ID NO 14, SEQ ID NO 15 and/or SEQ ID NO 16.

One exemplary embodiment provides a use of a nucleic acid or polynucleotide that prevents or reduces the expression of the gene that encodes the Snail1 protein in the preparation of a drug or pharmaceutical composition useful in the treatment of a chondrodysplasia process, where the nucleotide sequence is selected from the list that includes: a) an anti-sense nucleotide sequence specific to the gene or mRNA sequence of the Snail1 protein, b) a ribozyme specific to the mRNA of the Snail1 protein, c) an aptamer specific to the mRNA of the Snail1 protein, d) an interference RNA (siRNA or shRNA) specific to the mRNA of the Snail1 protein, and e) a microRNA (miRNA) specific to the Snail1 protein.

Another exemplary embodiment is characterized in that the siRNA of d) is an siRNA that binds to the Snail mRNA fragment included in SEQ ID NO: 17 or to another sequence that comprises the latter or a shorter fragment thereof.

Another exemplary embodiment is characterized in that the siRNA of d) is composed of a pair of nucleotide sequences, or a mixture thereof, belonging to the following group: -I: SEQ ID NO 11 and the complementary thereof, SEQ ID NO 12, -II: SEQ ID NO 13 and the complementary thereof, SEQ ID NO 14, and -III: SEQ ID NO 15 and the complementary thereof, SEQ ID NO 16.

An exemplary embodiment also includes a transgSnail1-ER transgenic mouse as described in Example 2.

### DESCRIPTION OF FIGURES

FIG. 1. Snail1 is expressed during embryonic bone development in the populations involved in the longitudinal growth thereof.

Images of embryonic bone sections wherein the presence of mouse Snail1 mRNA has been detected by means of the in situ hybridization technique. The upper panels (A-D) show the endogenous gene expression during development, first in the mesenchymal condensations and, subsequently, reduced to the hypertrophic condrocyte populations. The lower panels (E-I) compare their expression pattern with that of cell population marker molecules, and it can be observed that Snail1 is expressed at 18.5 days post-coitum (dpc) in the hypertrophic populations, the perichondrium and the osteoblasts. (J, K) detail of the growth plate of embryos at 18.5 dpc, which show the expression of Snail1 and FGFR3. In this and the following figures: wt, wild mouse; tg, transgenic mouse with inducible activation of Snail1; −TAM, without tamoxifen; +TAM, with tamoxifen.

FIG. 2. The long bones of embryos that express transgenic Snail1 are shorter.

(A-D) cartilage-bone staining that shows a reduction in the cartilage area (blue) at the expense of the populations of the growth plate in the bones of animals with activation of Snail1 induced by the administration of Tamoxifen. (E-H) histological sections which show that the shortening described above is at the expense of the proliferative chondrocyte populations.

FIG. 3. The presence of Snail1 in the growth plate inhibits cell proliferation.

(A-D) immunohistochemistry against PH3 to mark proliferating cells, where a drastic decrease in the number of proliferative cells can be observed in mice with an induced expression of Snail1. (E-H) in these same mice, the immunohistochemistry against STAT1 reveals an increase in the activation thereof (nuclear presence of the protein), which is accompanied by an increase in the levels of p21 mRNA (m). A detail of the endogenous co-expression of STAT1 and Snail1 under normal conditions in mouse bone can also be observed (I-L).

FIG. 4. Snail1 is sufficient for FGFR3 signaling in the bone.

The activation of FGFR3 signaling in mouse primary chondrocyte cultures (A-D), due to the transfection of the mutated versions of FGFR3 representative of human chondrodysplasias ACH and TDII (K644E and G380R, respectively), induces the activation of Snail1, evaluated by the mRNA levels thereof (E), which is accompanied by the activation of p21 (G), without transcriptional variations of STAT1 (F). Moreover, the sole activation of Snail by the addition of 4-OHT in these cultures (I) causes the phosphorylation of ERK1/2 and, therefore, activation of the MAPK pathway, which is responsible for the delayed differentiation of chondrocytes, and can be measured by the persistence of Sox9 expression therein (H). Mock, primary cultures transfected with the empty vector; wt, primary cultures transfected with normal human FGFR3; K644E, primary cultures transfected with human FGFR3 with mutation K644E, responsible for most human thanatophoric dysplasias; G380R, primary cultures transfected with human FGFR3 with mutation G380R.

FIG. 5. Snail1 is necessary for FGFR3 signaling in the bone.

The effects described in FIG. 4 are prevented by treating the cultures with Snail1 siRNA.

FIG. 6. There is a correlation between Snail1 expression levels and the severity of the achondroplasia phenotype in mice.

The phenotypes were divided into three groups, low (25%, A, B, G), medium (60%, C, D, H) and high (15%, E, F, I). The severity of the phenotype is related to the expression levels of Snail1.

FIG. 7. The FGFR3 mutation responsible for thanatophoric dysplasia in humans activates the expression of Snail1.

Snail1 mRNA levels are increased in cartilage from a human thanatophoric fetus (severe form of achondroplasia related to the constitutive activation of FGFR3) with respect to one that has no skeletal development problems (a). The thanatophoric fetus exhibits the mutation described as constitutively active of the receptor (b). N, sample obtained from a fetus without bone problems; T, sample obtained from a fetus with thanatophoric dysplasia.

### DETAILED DESCRIPTION

The Snail1 gene is involved in transducing the signaling mediated by FGF receptor 3 (FGFR3), which contributes to the occurrence of chondrodysplasia. In order to determine whether the Snail1 gene is involved in this signaling pathway, its relative expression patterns were studied during embryonic development in mice, when the different processes wherein they are involved during the formation of mature bone in healthy mice take place (Example 1). The data show that the expression patterns coincide in embryonic stages, since the expression peak of Snail1 appears in the cell population that expresses FGFR3. These data are compatible with the fact that FGFR3 induces the expression of the Snail1 gene in vivo.

Also studied were the effects of the pathological activation of Snail1 on the bone development of transgenic mice with inducible expression of the Snail1 gene (Example 2). In this way, the presence of Snail1 has been related to the interruption of the proliferation of chondrocytes in transgenic mice wherein Snail1 was artificially activated (transgenic transgSnail1-ER mouse); at the same time, it was demonstrated that the aberrant activation of Snail1 can be sufficient to reproduce a chondrodysplasia phenotype. The development and characterization of this animal model, the transgSnail1-ER mouse, allows for the availability of animal models (this transgenesis process can be extrapolated to the development of other mammalian animals, including non-human primates, by those of ordinary skill in the art) wherein therapeutic compounds can be tested and characterized for use in the treatment of veterinary or human chondrodysplasia processes, which did not exist until now.

Thus, it was observed that Snail1 can repress cell proliferation in vivo, which can induce morphological defects in the growth plate in these bones (shorter bones). The changes described herein are reminiscent of those observed following experimental induction of chondrodysplasia in genetically modified mice that expressed mutated versions of FGFR3, which lead to the constitutive activation thereof.

Likewise, Snail1 has been associated with the FGFR3 signaling pathway by means of in vitro experiments in primary chondrocyte cultures obtained from mouse embryonic bones, which shows that the FGFR3-mediated signaling can activate the expression of Snail1 (Example 3).

On the other hand, the activation of the Snail1 gene was studied in samples from patients with chondrodysplasia (Example 4). In this regard, in an unborn child with thanatophoric dysplasia, the mutation in FGFR3 responsible for this disease (severe form of chondrodysplasia with an always lethal evolution) was confirmed. It was observed that this mutation in FGFR3, which produces its constitutive activity, can cause an aberrant activation of the Snail1 gene.

Finally, the interference RNA (“small interference”, siRNA) experiments performed on primary cultures from mouse fetal bones showed that preventing the Snail1 function can be sufficient to hinder FGFR3-mediated signaling in chondrocytes, reversing the interruption of the cell cycle (mediated by the expression of p21) and the activation of the MAPK pathway (recognized by the phosphorylation of ERK1/2), even in the presence of constitutively active FGFR3 (Example 3). Therefore, Snail1 is involved in FGFR3 signaling. The above-mentioned polynucleotides can be used in a gene therapy process wherein, using any technique or method known to those of ordinary skill in the art, the integration thereof in the cells of a human patient is made possible.

If de-regulated Snail activity in embryonic cartilage is in itself capable of inducing achondroplasia, the presence of Snail1 in stages where it is normally repressed could be considered to be a marker of the achondroplasia phenotype of the type associated with FGFR3—without limitation, achondroplasia (ACH), thanatophoric dysplasia (TD) and hypochondroplasia (HCH)—and, therefore, its aberrant expression could be used as a diagnosis for any phenotype caused by the gain-of-function of FGFR3. On the other hand, if the activation of Snail1 is sufficient to induce the characteristics of the achondroplasias related to mutations that generate an increase in FGFR3 activity, i.e. there is a direct relation—and not only a temporary association—between Snail1 gene activity and the etiopathogeny of this disease, the inhibition thereof, e.g. the gene inhibition thereof, would become a form of anti-dysplasia therapy and Snail1 could be very useful in the identification of new anti-chondrodysplasia drugs. These therapeutic approaches are based on the use of inhibitory compounds or agents of the Snail1 protein. In fact, cases of chondrodysplasia of this type might appear even in the absence of mutations in FGFR3 by the sole pathological activation of Snail1 during the development of the cartilage-bone system.

Experiments concerning Snail1 expression in mouse embryos showed that there is a direct correlation between expression levels and the severity of the achondroplasia phenotype in mice, which indicates that Snail1 behaves as a risk factor and confirms its significance as a potential target for anti-achondroplasia therapies (Example 4).

The aberrant presence of Snail1 in embryonic cartilage (at a time when, under normal conditions, it no longer occurs) can induce achondroplasia, regardless of the signal that has induced this pathological presence of Snail1. The inhibition of Snail1 by siRNA can prevent the interruption of the proliferation of chondrocytes and the phosphorylation of ERKs even in the presence of constitutive FGFR3 activity. Therefore, the inhibition of Snail1 in the growth plate can prevent the effects of the gain-of-function of FGFR3 induced by any mechanism or the achondroplasias generated by the aberrant presence of Snail1 independent from FGFR3.

Exemplary embodiments disclosed herein include a method for identifying a chondrodysplasia process based on the identification of the presence of Snail1 in a biological sample, which can comprise the following steps:

a) identifying the presence of Snail1 in a biological sample of osseous origin, and

b) comparing the presence of Snail1 observed in a) to its absence in a control sample, where its presence is indicative of the existence of chondrodysplasia.

As used herein, the term “chondrodysplasia process” refers to a disease with a chondrodysplasia phenotype, in one exemplary embodiment human, wherein the biological action of Snail 1 is a cause of the disease, whether or not it is accompanied by an anomalous activation of FGFR3—such as, for example and without limitation: achondroplasia (ACH), thanatophoric dysplasia (TD) and hypochondroplasia (HCH). This identification of Snail1 in a biological sample of veterinary or medical origin from animals or human subjects can be extracted therefrom and, subsequently, the presence or absence of Snail1 therein identified ex vivo, which can be correlated with the diagnosis of a chondrodysplasia process in the subject and can allow for the definition and execution of a therapeutic approach (without limitation, for veterinary or medical purposes).

As used herein, the term “Snail1 gene” or “Snail1 protein” refers both to the Snail1 gene or protein, of different animal origins, in one exemplary embodiment mouse or human (for example, mouse Snail1: SEQ ID NO 1 and SEQ ID NO 2, or human Snail1: SEQ ID NO 3 and SEQ ID NO 4, respectively), and to any nucleotide or amino acid (aa) sequence respectively analogous thereto. In the sense used in this description, the term “analogous” includes any nucleotide or amino acid sequence that can be isolated or constructed on the basis of the nucleotide or amino acid sequences shown in this specification, for example, by the introduction of conservative or non-conservative nucleotide or amino acid substitutions, including the insertion of one or more nucleotides or amino acids, the addition of one or more nucleotides or amino acids at any of the ends of the molecule, or the deletion of one or more nucleotides or amino acids at any end or in the interior of the sequence, which is an encoding sequence or a peptide with an activity similar to the Snail1 sequences described herein, i.e., capable of inducing chondrodysplasia.

In general, an analogous nucleotide or amino acid sequence is substantially homologous to the amino acid sequence previously discussed. In the sense used in this description, the expression “substantially homologous” means that the nucleotide or amino acid sequences in question have a degree of identity of at least 40%, in one exemplary embodiment of at least 85%, or more in one exemplary embodiment of at least 95%.

Exemplary embodiments disclosed herein include an identification method wherein the identification of Snail1 of a) relates to the human form of Snail1 (hSnail1, whether the identification is in the form of a gene transcript (mRNA) or the protein form of the gene; SEQ ID NO 3 and SEQ ID NO 4).

These analyses designed to identify Snail1 expression levels can be performed by one of ordinary skill in the field of biomedicine, based on the information disclosed herein and in the state of the art, by means of different techniques including those described in, without limitation Sambrook et al., 1989; Lambolez and Rossier, 2000; Egger et al., 2000; Folz and Nepluev, 2000, and Pfaffl, 2001 all of which are incorporated by reference herein for their relevant teachings.

Exemplary embodiments disclosed herein include methods for identifying a chondrodysplasia process wherein the identification of Snail1 can be performed using specific Snail1 protein antibodies, in one exemplary embodiment hSnail1 as described by Franci et al., 2006 which is incorporated by reference herein. The antibodies can be monoclonal or polyclonal.

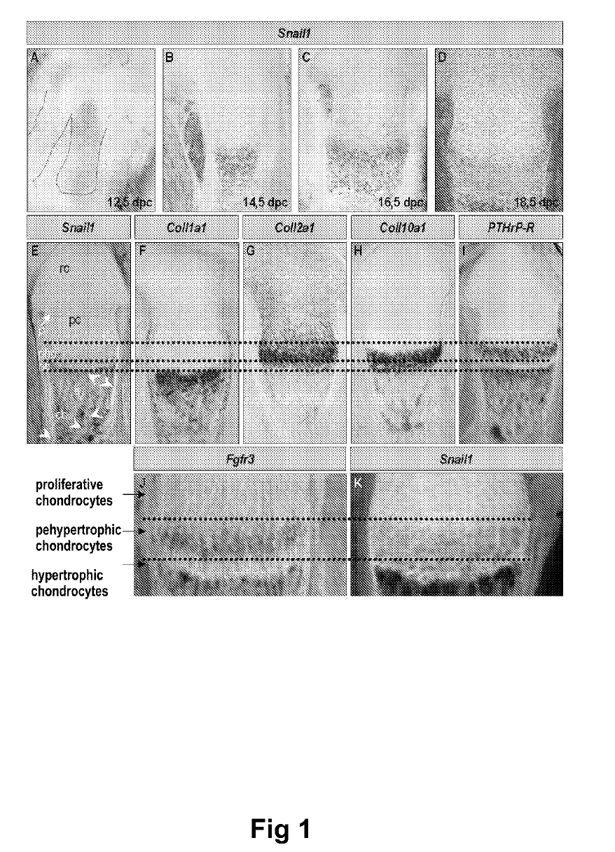

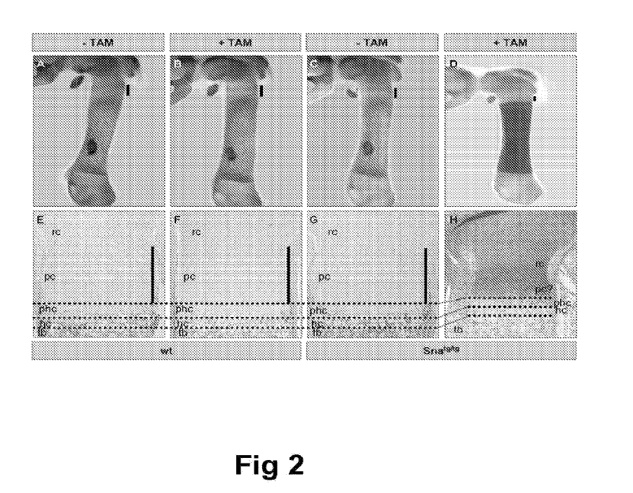

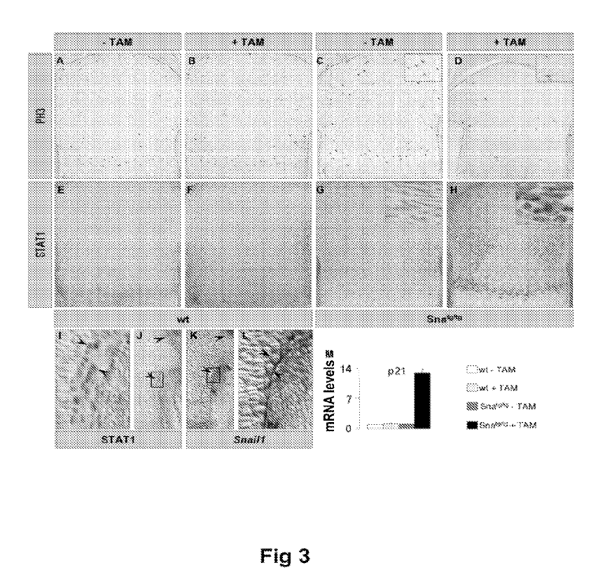

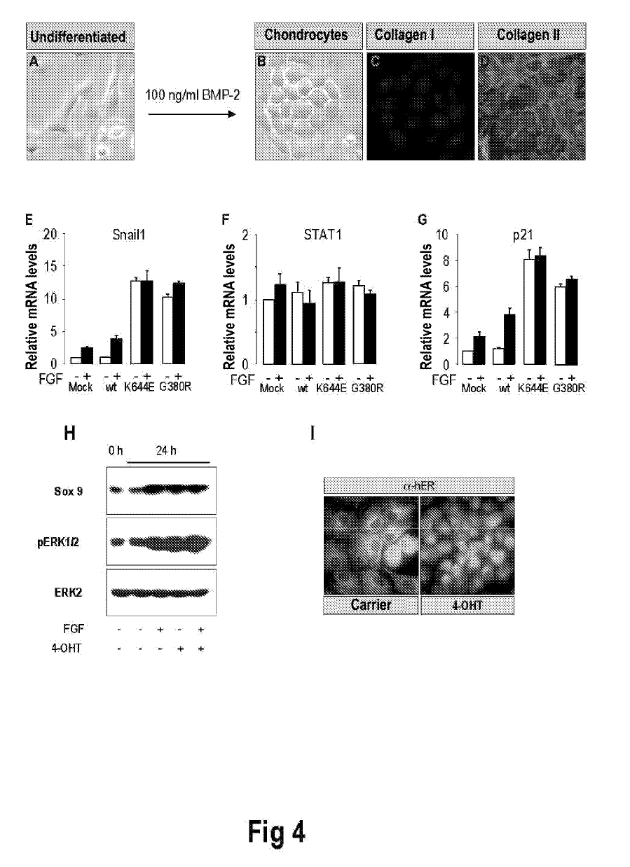

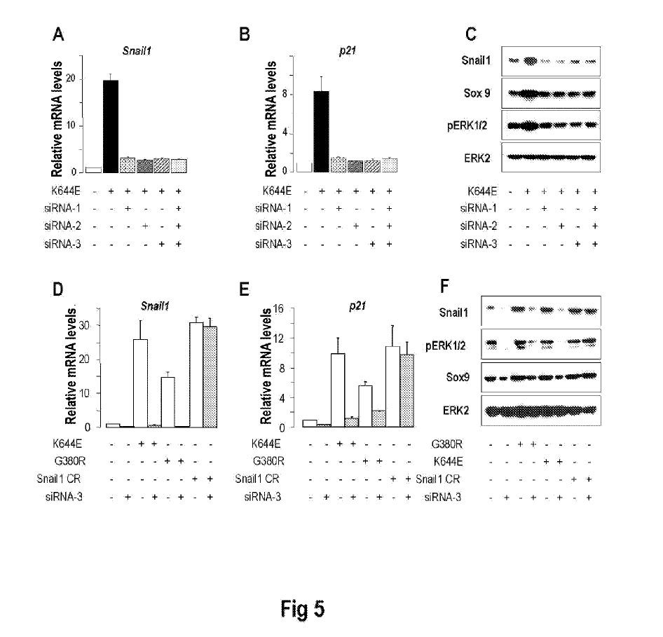

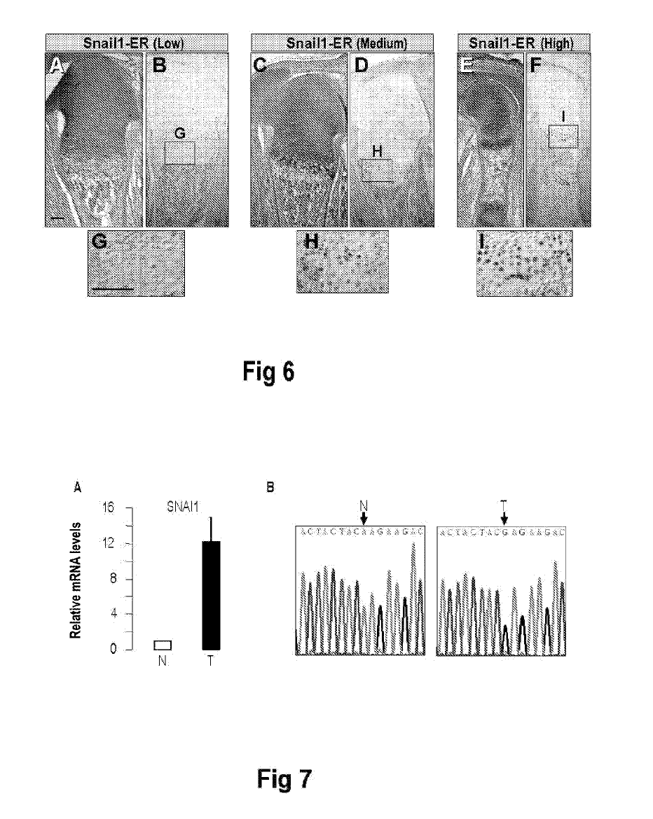

Exemplary embodiments disclosed herein include methods for identifying a chondrodysplasia process wherein the identification of Snail1 can be performed by in situ hybridization with a Snail1 precursor (see FIG. 1).

Exemplary embodiments disclosed herein include methods for identifying a chondrodysplasia process wherein the identification of Snail1 can be performed by RT-PCR of a Snail1 gene precursor (Example 3, FIG. 4). This method can be based on the extraction of polyA+RNA from a biological sample of osseous origin and a control tissue, and amplification of the Snail1-encoding sequence with suitable primer oligonucleotides as described in, for example and without limitation, Boutet et al., 2006 which is incorporated by reference herein for its teaching regarding the same. This diagnostic method for chondrodysplasia can also be performed using Snail1 as the sole marker or jointly with other chondrodysplasia markers; for example, as a part of a biological expression microarray, either in, without limitation, gene form—from mRNA—or in protein form.

Exemplary embodiments disclosed herein include methods for identifying and evaluating the activity of inhibitory compounds of the Snail1 protein useful in the treatment of chondrodysplasia, hereinafter method for identifying compounds, which can comprise the following steps:

a) placing a biological system with an expression of Snail1 that produces chondrodysplasia in contact with the candidate compound of this method, and incubation under suitable conditions,

b) determining a parameter that is indicative of the chondrodysplasia process, and

c) identifying a compound inhibitory of Snail1 protein activity when a reduction in the chondrodysplasia parameter is observed.

Exemplary embodiments disclosed herein include methods for identifying compounds where the biological system of point a) can be a transgenic animal wherein the expression of the Snail1 protein can be inducible, in a constant or conditional manner, and the expression thereof can cause chondrodysplasia. A particular embodiment of the method for identifying compounds is one where the transgenic animal can be the transgenic mouse described in Example 2, the transgSnail1-ER mouse.

Exemplary embodiments disclosed herein include biological systems for performing methods for identifying compounds described herein, in one exemplary embodiment a transgenic animal, in one exemplary embodiment a mammal, and, in one exemplary embodiment, a non-human primate, where the expression of the Snail1 protein can be inducible, in a constant or conditional manner, and the expression thereof can cause chondrodysplasia. A particular embodiment of the non-human mammalian animal is the transgenic mouse described in Example 2, the transgSnail1-ER mouse.

Exemplary embodiments disclosed herein include uses of a compound or agent inhibitory of Snail1 protein activity, hereinafter use of an inhibitory compound, in the preparation of a drug or pharmaceutical composition useful in the treatment of a chondrodysplasia process, in one exemplary embodiment human or veterinary.

As used herein, the term “inhibitory or antagonist compound/agent” refers to a molecule which, when it is bound to or interacts with the Snail1 protein (for example and without limitation, SEQ ID NO 2 or SEQ ID NO 4), or with functional fragments thereof, reduces or eliminates the intensity or the duration of the biological activity of the protein. This definition includes, furthermore, those compounds that prevent or reduce the expression of the Snail protein encoding genes (for example and without limitation, SEQ ID NO 1 or SEQ ID NO 3), i.e. that prevent or reduce gene transcription, mRNA maturation, mRNA translation and post-translational modification. An inhibitory agent can be composed of a peptide, a protein, a nucleic acid or polynucleotide, a carbohydrate, an antibody, a chemical compound or any other type of molecule that reduces or eliminates the effect and/or function of the Snail1 protein.

For illustrative purposes, a polynucleotide can be, without limitation, a polynucleotide that encodes a Snail1 protein gene or an mRNA sequence specific anti-sense nucleotide sequence, or a polynucleotide that encodes a Snail1 protein mRNA specific ribozyme, or a polynucleotide that encodes a Snail1 protein mRNA specific aptamer, or a polynucleotide that encodes a Snail1 protein mRNA specific interference RNA (“small interference RNA” or sRNA, or an shRNA) or a microRNA (miRNA).

The above-mentioned polynucleotides can be used in a gene therapy process which, by means of any technique or procedure known to those of ordinary skill in the art, allows for the integration thereof in the cells of a human patient. This objective can be achieved by administering a gene construct comprising one of the above-mentioned polynucleotides to these bone or cartilage cells in order to transform the cells, allowing for their expression in the interior thereof in such a way that expression of the Snail protein can be inhibited. Advantageously, the gene construct can be included within a vector, such as, for example, an expression vector or a transfer vector.

As used herein, the term “vector” refers to systems used in the process of transferring an exogenous gene or an exogenous gene construct inside the cell, thereby allowing for the transport of exogenous genes and gene constructs. Vectors can be non-viral or viral vectors and the administration thereof can be prepared by a person of ordinary skill in the art on the basis of the needs and specificities of each case.

Exemplary embodiments disclosed herein include uses of an inhibitory compound of Snail1 wherein the inhibitory compound can be a nucleic acid or polynucleotide that can prevent or reduce the expression of the gene that encodes the human Snail1 protein and can include a nucleotide sequence selected from:

a) an anti-sense nucleotide sequence specific to the gene or mRNA sequence of the Snail1 protein,

b) a ribozyme specific to the mRNA of the Snail1 protein,

c) an aptamer specific to the mRNA of the Snail1 protein,

d) an interference RNA (siRNA or shRNA) specific to the mRNA of the Snail1 protein, and

e) a microRNA (miRNA).specific to the Snail1 protein.

Exemplary inhibitory compounds include, without limitation, antisense oligonucleotides described in, without limitation, US Patent Publication No. US20060003956 and Kajita et al., 2004 or siRNAs that inhibit the expression of Snail1 such as those described in without limitation, Peinado et al., 2005 and Tripathi et al., 2005 all of which are incorporated by reference herein. Additionally, any published nucleotide sequences or those published in the future that inhibit the expression of Snail1 are incorporated as embodiments herein, as potentially useful therapeutic compounds for the preparation of drugs designed to treat a chondrodysplasia process. Gene inhibition techniques and, more specifically, transport of compounds including, without limitation, antisense oligonucleotides, siRNA, ribozymes or aptamers—can be performed using, without limitation, liposomes, nanoparticles or other carriers that increase the success rate of the transfer to the interior of the cell, in one exemplary embodiment the cell nucleus (see, without limitation, Lu and Woodle, 2005 and Hawker and Wooley, 2005 which are incorporated by reference herein). In principle, Snail1 mRNA translation inhibitors can be used which bind both to the encoding region and/or the non-encoding region, for example, in front of the 3′ non-encoding area.

Thus, one exemplary embodiment includes the use of an siRNA of d) wherein the siRNA in one exemplary embodiment can bind to the Snail mRNA gatgcacatccgaagccac (SEQ ID NO 17) fragment sequence or to another sequence that comprises the latter or a shorter fragment thereof (see US Patent Publication No. US20060003956; the use of the siRNAs disclosed therein are incorporated by reference herein).

Another exemplary embodiment is the use of an siRNA of d) wherein the siRNA can be composed of a pair of nucleotide sequences, or a mixture thereof, belonging to the following groups:

(SEQ ID NO 11)
I: 5′-CGG AAG AUC UUC AAC UGC AAA UAU U-3′,
(SEQ ID NO 12)
complementary: 5′-AAU AUU UGC AGU UGA AGA
UCU UCC G-3′.
(SEQ ID NO 13)
II: 5′-CAA ACC CAC UCG GAU GUG AAG AGA U-3′,
(SEQ ID NO 14)
complementary: 5′-AUC-UCU UCA CAU CCG AGU GGG
UUU G-3′,
and
(SEQ ID NO 15)
III: 5′-CAG CUG CUU CGA GCC AUA GAA CUA A-3′,
(SEQ ID NO 16)
complementary: 5′-UUA GUU CUA UGG CUC GAA GCA
GCU G-3′.

Pairs I and II can bind to the encoding region of the Snail1 mRNA, whereas pair III can bind to the 3′ non-encoding region. The three pairs of siRNA sequences specified were active, and the image of the inhibition of Snail1 and p21 levels shown is representative of the results obtained by any of the three pairs of siRNAs (FIG. 5), either by themselves or in combination.

Nucleotide sequences a)-e) mentioned above can prevent gene expression in mRNA or mRNA expression in the Snail1 protein, and, therefore, can destroy its biological function, and can be developed by a person of ordinary skill in the field of genetic engineering on the basis of the existing knowledge about transgenesis and gene expression destruction in the state of the art (see, for example, Clarke, 2002; US Patent Publication No. US20020128220; Miyake et al., 2000; Puerta-Fernandez et al., 2003; Kikuchi et al., 2003; Reynolds et al., 2004 all of which are incorporated by reference herein).

On the other hand, these compounds that inhibit the activity of Snail1 proteins can have a varied origin, such that they can be of natural origin (for example, and without limitation, vegetable, bacterial, viral or animal origin, or from eukaryotic microorganisms) or of synthetic origin.

Exemplary embodiments disclosed herein include pharmaceutical compositions useful in the treatment of a chondrodysplasia process which can comprise a therapeutically effective quantity of an inhibitory compound or agent of the Snail1 protein, jointly with, optionally, one or more pharmaceutically acceptable adjuvants and/or carriers.

An exemplary embodiment is a pharmaceutical composition wherein the inhibitory compound can be a nucleic acid or polynucleotide which can prevent or reduce the expression of the gene that encodes the human Snail1 protein and can include a nucleotide sequence selected from:

a) an anti-sense nucleotide sequence specific to the gene or mRNA sequence of the Snail1 protein,

b) a ribozyme specific to the mRNA of the Snail1 protein,

c) an aptamer specific to the mRNA of the Snail1 protein,

d) an interference RNA (iRNA including without limitation, sRNA and shRNA) specific to the mRNA of the Snail1 protein, and

e) a microRNA (miRNA) specific to the Snail1 protein.

Another exemplary embodiment is the pharmaceutical composition wherein the Snail1 inhibitor is an sRNA that in one exemplary embodiment binds to the Snail mRNA gatgcacatccgaagccac (SEQ ID NO 17) fragment sequence or to another sequence that comprises the latter or a shorter fragment thereof (see US Patent Publication No. US20060003956; the use of the siRNAs disclosed therein are incorporated by reference herein).

Another exemplary embodiment is the use of a pharmaceutical composition wherein the Snail inhibitor is an siRNA composed of a pair of nucleotide sequences, or a mixture thereof, belonging to the following group:

(SEQ ID NO 11)
I: 5′-CGG AAG AUC UUC AAC UGC AAA UAU U-3′,
(SEQ ID NO 12)
complementary: 5′-AAU AUU UGC AGU UGA AGA
UCU UCC G-3′.
(SEQ ID NO 13)
II: 5′-CAA ACC CAC UCG GAU GUG AAG AGA U-3′,
(SEQ ID NO 14)
complementary: 5′-AUC UCU UCA CAU CCG AGU
GGG UUU G-3′,
and
(SEQ ID NO 15)
III: 5′-CAG CUG CUU CGA GCC AUA GAA CUA A-3′,
(SEQ ID NO 16)
complementary: 5′-UUA GUU CUA UGG CUC GAA
GGA GCU G-3′ .

The pharmaceutically acceptable adjuvants and carriers that can be used are the adjuvants and carriers known by those of ordinary skill in the art and usually used in the preparation of therapeutic compositions.

In the sense used in this description, the expression “therapeutically effective quantity” refers to the quantity of the inhibitory agent or compound of Snail1 protein activity calculated to produce the desired effect and, in general, can be determined, amongst other factors, by the compounds' characteristics, including the patient's age, condition, the severity of the alteration or disorder, and the administration route and frequency.

In a particular exemplary embodiment, the therapeutic composition can be prepared in solid form or in aqueous suspension in a pharmaceutically acceptable diluent. The therapeutic compositions can be administered by any suitable administration route; to this end, the composition can be formulated in the pharmaceutical form suitable for the chosen administration route. In a particular exemplary embodiment, administration of the therapeutic composition can be performed by, without limitation, parenteral route, by oral route, by intraperitoneal route, by subcutaneous route, etc. A review of the different pharmaceutical forms to administer drugs and the necessary excipients to obtain them can be found, for example, in Faulí i Trillo, 1993.

Exemplary embodiments disclosed herein include uses of pharmaceutical compositions described herein in a treatment method for a mammal, in one exemplary embodiment a human being, suffering from a chondrodysplasia process which consists in administering the therapeutic composition that inhibits the chondrodysplasia process.

Exemplary embodiments disclosed herein include uses of pharmaceutical compositions disclosed herein wherein the chondrodysplasia process caused by the biological action of Snail1 is accompanied by an anomalous activation of FGFR3, which, for non-limiting illustrative purposes belongs to the following non-limiting group: achondroplasia (ACH), thanatophoric dysplasia (TD) and hypochondroplasia (HCH).

Exemplary embodiments disclosed herein include uses of pharmaceutical compositions disclosed herein wherein the chondrodysplasia process caused by the biological action of Snail1 is not accompanied by anomalous FGFR3 activation.

### EXAMPLES

### Example 1

### Snail1 is Expressed During Embryonic Bone Development in the Populations Involved in the Longitudinal Growth Thereof

The presence of Snail1 mRNA in embryonic mouse bones was detected by means of an in situ hybridization technique. The mouse embryos were obtained from the C57xCBA strain and their ages, established in days post-coitum (dpc), were determined by considering the day, where the vaginal plug is seen as day 0.5. The bones were desiccated at stages between 12.5 dpc and 18.5 dpc, respectively, and fixated in 4% paraformaldehyde in PBS/DEPC overnight. Subsequently, they were soaked in gelatin and cut with a vibratome, to obtain 50-m sections. The ISH in gelatin sections were performed as described in Blanco et al., 2002 (incorporated by reference herein), using RNA probes labeled with DIG-11-UTP. Following hybridization, the sections were processed as described in Cano et al., 2000 (incorporated by reference herein).

Thus, it was shown that the endogenous expression of the Snail1 gene takes place during development; first in the mesenchymal condensations and, subsequently, reduced to the hypertrophic chondrocyte populations, the perichondrium, and the osteoblasts (FIG. 1).

### Example 2

### Snail1-ER Transgenic Mice Present Alterations in Bone Growth

The pcDNA3-Snail1 plasmid, corresponding to the complete sequence of mouse Snail1 cDNA inserted in the pcDNA3 plasmid (Invitrogen; Cano et al., 2000 (incorporated by reference herein)), was used. pcDNA3-Snail-ER corresponds to the Snail1 encoding sequence bound to a mutated version of the binding domain to the human estrogen receptor agonist that recognizes the 4-OH-Tamoxifen synthetic ligand.

The Snail1-ER transgene was designed as previously described (Locascio et al., 2002 (incorporated by reference herein)) and a transgenic mouse (transgSnail1-ER mouse) was generated for this construct in accordance with standard procedures (Hogan et al., 1994 (incorporated by reference herein)). For this study, an animal line was selected in which the transgenic protein expression was ubiquitous in the embryo. In this model, although the Snail1-ER protein is constitutively expressed, its function as a transcription factor only develops when the protein is translocated to the nucleus following treatment with tamoxifen. The transgene is detected from the DNA taken from the animals' tail by PCR. The protein's sub-cellular location was analyzed by immunohistochemistry using an anti-human estrogen receptor antibody. The same antibody was used to evaluate the quantity of Snail1-ER protein in the different tissues obtained from the transgenic mice by Western Blot. The tamoxifen (Sigma) was first dissolved in ethanol (10% of the final volume) and subsequently in corn oil (Sigma) in order to obtain a final concentration of 30 mg/ml. Two consecutive intraperitoneal injections of tamoxifen are administered to pregnant females at days 12.5 and 14.5 dpc. The dose used was 57 g/g of weight of the female.

Subsequently, in order to perform the cartilage-bone staining experiments, the desiccated embryos and the post-natal mice of the transgSnail1-ER transgenic mouse were fixated in 10% buffered formalin at ambient temperature for a minimum of 2 days. They were eviscerated and their skin removed. They were washed in water for between 2 hours and 2 days, depending on the size of the specimen. They were stained with an alcian blue solution (Sigma A5268) for 12-48 hours, in order to stain the cartilages. After washing the specimens in absolute Ethanol for at least two days, they were rehydrated and incubated in trypsin (Sigma T4799) until complete maceration of the tissue. They were transferred to an alizarin red S solution (Sigma A5533) in 0.5% KOH until the bones were stained red. The specimens were washed in consecutive solutions of 3:1, 1:1 and 1:3 0.5% KOH/glycerin solutions, and preserved in pure glycerin.

The study of the bones of transgSnail1-ER transgenic mice embryos showed that these were shorter than those that did not overexpress Snail1 (FIG. 2), due to a reduction in the cartilage area at the expense of the proliferative chondrocyte populations. Furthermore, the expression of Snail1 was associated with an inhibition of the proliferation of proliferative chondrocytes and accompanied by the translocation of STAT1 to the nucleus and an increase in the expression of p21 (FIG. 3).

In order to perform the immunohistochemistry assays, bone sections were analyzed. In detail, the sections were obtained by means of cuts in microtomes and treated for 30 minutes in 1 mM EDTA at 100° C., in order to unmask the antigen. After removing the paraffin residues and hydrating, they were permeabilized for 30 minutes in 0.5% Triton X-100 in PBS at ambient temperature and, subsequently, blocked for 1 hour in 0.1% Tween, 10% FCS in PBS. The preparations were incubated with the primary antibodies in 0.1% Tween-20, 1% FCS in PBS for 16 hours at 4° C., and with the secondary antibodies in 0.1% Tween-20, 1% FCS in PBS for 2 hours at ambient temperature. The preparations were mounted on Mowiol and preserved protected from light at 4° C. until they were viewed in a microscope.

The mRNA levels of p21 were analyzed by RT-PCR. In this regard, and in the rest of the mRNA analyses, the quantitative PCR was performed using an ABI PRISM® 7000 quantitative PCR machine following the Syber-Green® method. The expression levels were calculated in accordance with the Ct method, using GAPDH as a normalizer and the wild mouse levels without any treatment or transfection as a calibrator. For these studies, the following primer sequences (all 5′-3′) were used: mGadph, CTGAGCAAGAGAGGCCCTATCC (SEQ ID NO 5) and CTCCCTAGGCCCCTCCTGTT (SEQ ID NO 6); mP21, AGGAGCCAGGCCAAGATGGT (SEQ ID NO 7) and GCTTTGACACCCACGGTATTCA (SEQ ID NO 8); mSnail1, CCACACTGGTGAGAAGCCATTC (SEQ ID NO 9) and TCTTCACATCCGAGTGGGTTTG (SEQ ID NO 10).

### Example 3

### Snail1 is Sufficient and Necessary for FGFR3 Signaling in Chondrocytes

The primary chondrocyte cultures were obtained from bone of the back legs of C57 animal embryos at 14.5 dpc, which were desiccated in culture medium (-MEM, 1% BSA, 0.1% L-Glutamine, 0.1% penicillin/streptomycin). On the following day, they were trypsinized and treated with collagenase in DMEM and 10% serum. They were cultured in a medium (50% F-12, 50% DMEM, 10% FCS, 0.1% L-Glutamine, 0.1% penicillin/streptomycin) with a cell density of 1.5×10⁶ cells/P100. After 5 days in culture, the differentiation was started with BMP-2 (FIG. 4).

The activation of FGFR3 in mouse primary chondrocyte cultures with FGF induced the activation of Snail1, evaluated by means of the increase in the mRNA levels thereof (FIG. 4E), which was accompanied by the activation of p21 (FIG. 4G) and the activation of the MAPK signaling pathway; the phosphorylation levels of ERK1/2 and the levels of Sox9 were increased (FIG. 4H), as measured by the Western Blot technique; moreover, the expression of the mutant form of FGFR3 causes the constitutive expression of Snail1, regardless of the presence of ligand (FIG. 4E). The sole activation of Snail1 induced by the administration of 4-OHT in primary chondrocyte cultures from the transgenic mouse (transgSnail1-ER mouse) causes the activation of the MAPK signaling pathway, recognized by the increase in the phosphorylation levels of ERK1/2 and the levels of Sox9 (FIG. 4H). As an example of a method to control the levels of Snail1—and a model to treat a chondrodysplasia process—, a regulation assay of the expression of Snail1 was performed using specific siRNAs, and it was observed that the siRNAs prevented the expression of Snail1 and the increased expression of p21 and Sox9 and the phosphorylation of ERK1/2 (FIG. 5). Three different siRNAs were used, two against encoding region sequences and one against the 3′ non-encoding region, in order to ensure the specificity of the results. The sequences of these siRNAs were the following:

(SEQ ID NO 11)
I (encoding): 5′-CGG AAG AUC UUC AAC UGC AAA
UAU U-3′,
(SEQ ID NO 12)
complementary: 5′-AAU AUU UGC AGU UGA AGA UCU
UCC G-3′.
(SEQ ID NO 13)
II (encoding): 5-CAA ACC CAC UCG GAU GUG AAG
AGA U-3′,
(SEQ ID NO 14)
complementary: 5′-AUC-UCU UCA CAU CCG AGU
GGG UUU G-3′,
and
(SEQ ID NO 15)
III (3′ non-encoding area): 5′-CAG CUG CUU CGA
GCC AUA GAA CUA A-3′,
(SEQ ID NO 16)
complementary: 5′-UUA GUU CUA UGG CUC GAA GCA
GCU G-3′.

The three pairs of siRNA sequences specified were active, and the image of the inhibition of the levels of Snail1, p21, Sox9 and phosphorylation of ERK1/2 shown is representative of the results obtained by any of the three pairs of siRNAs (FIG. 5), either by themselves or in combination.

### Example 4

### There is a Correlation Between Snail1 Expression Levels and the Severity of the Achondroplasia Phenotype in Mice

The bones of mouse embryos at 18.5 dpc were obtained as explained in Example 1. The sub-cellular location of the Snail1-ER fusion protein was analyzed as explained in Example 2 and the histological staining of the samples was performed as specified in the section about materials and methods. It was observed that the greater the presence of active Snail1 protein in the bones (FIG. 6F, I), the more aggressive the achondroplasia phenotype, with shorter, more curved bones (FIG. 6E).

### Example 5

### The Mutation of FGFR3 Responsible for Thanatophoric Dysplasia in Humans Activates the Expression of Snail1

Snail1 mRNA levels are increased in cartilage from a human thanatophoric fetus (severe form of achondroplasia related to the constitutive activation of FGFR3) as opposed to one that has no skeletal development problems (FIG. 7A). The thanatophoric fetus exhibits the mutation described as constitutively active of the receptor (FIG. 7B).

### Materials and Methods

Histological staining. The sections were immersed in Haematoxylin (Harris Hematoxylin solution modified, Sigma HHS-16), for 1 minute, washed in tap water, and subsequently immersed in Eosin (Eosin Y Counterstain Solution, 0.5% Aqueous, Sigma HT110-2-32), for 30 seconds. After being washed in water, they were once again dehydrated, passed through Histoclear and mounted with Depex (BDH Laboratories) in order to be analyzed in the optical microscope.

Western Blot analysis. Extraction of total proteins from the cell cultures. The cells were washed with PBS and lysed with 150 l of RIPA buffer. The lysates were incubated for 30 minutes at 4° C. and centrifuged at 16,000 rpm for 10 minutes; it was assumed that the supernatant contained all the soluble proteins. The concentration of the extracts was measured by means of the Bradford method. The supernatants were mixed at 50% with 2× loading buffer and subject to electrophoresis in 8% agarose gel.

Transfer. The proteins were transferred to a nitrocellulose membrane in a mini-transblot cell at 110 V for 25 minutes.

Labeling with antibodies. The membranes were blocked with 3% skim milk in TBS for 1 hour at ambient temperature and subsequently incubated with the corresponding primary antibodies diluted in 3% skim milk in TBS for 16 hours at 4° C. After washing the membranes in TBS, they were incubated for 1 hour at ambient temperature with the corresponding peroxidase-coupled secondary antibodies. After washing the excess antibody, the membranes were developed by chemoluminescence and exposed on film. The total ERK-2 protein was used as a loading control.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

Boutet A, De Frutos C A, Maxwell P H, Mayol M J, Romero J and Nieto M A (2006) Snail activation disrupts tissue homeostasis and induces fibrosis in the adult kidney. EMBO J. 25:5603-5613.
Blanco M J, Moreno-Bueno G, Sarrio D, Locascio A, Cano A, Palacios J and Nieto M A (2002) Correlation of Snail expression with histological grade and lymph node status in breast carcinomas. Oncogene, 21:3241-3246.
Cano A, Pérez-Moreno M A, Rodrigo I, Locascio A, Blanco M J, del Barrio M G, Portillo and Nieto M A (2000) The transcription factor snail controls epithelial-mesenchymal transitions by repressing E-cadherin expression. Nat Cell Biol., 2:76-83.
Clarke, A R (2002) Transgenesis Techniques. Principles and Protocols, 2^{a} Ed. Humana Press, Cardiff University.
Faulí i Trillo C (1993) “Tratado de Farmacia Galénica”, Luzán 5, S. A. Ediciones, Madrid.
Franci C, Takkunen M, Dave N, Alameda F, Gómez S, Rodríguez R et al (2006) Expression of Snail protein in tumor-stroma interface. Oncogene, 25:5134-5144.
Folz R J and Nepluev I (2000) Poly(A) cDNA-specific (PACS) RT-PCR: a quantitative method for the measurement of any poly(A)-containing mRNA not affected by contaminating genomic DNA. Biotechniques, 29, 762, 764-5, 766-8.
Hawker C J and Wooley K L (2005) The convergence of synthetic organic and polymeric chemistries. Science 19 (309): 1200-5.
Hogan B, Beddington R, Constantini F, Lacy E (1994) ‘Manipulating the mouse embryo. A Laboratory Manual.’ (Cold Spring Harbor Laboratory Press)
Kajita M, McClinic K N, Wade P A (2004) Aberrant expression of the transcription factors snail and slug alters the response to genotoxic stress. Mol Cell Biol. 24(17): 7559-66.
Kikuchi K, Umehara T, Fukuda K, Hwang J, Kuno A, Hasegawa T and Nishi-kawa S (2003) RNA aptamers targeted to domain II of Hepatitis C virus IRES that bind to its apical loop region. J. Biochem. 133, 263-270.
Lambolez B and Rossier J (2000) Quantitative RT-PCR. Nat Biotechnol, 18, 5.
Locascio A, Vega S, de Frutos C A, Manzanares M, Nieto M A (2002) Biological potential of a functional human SNAIL retrogene. J Biol Chem 277, 38803-9.
Lu P V and Woodle M C. In vivo application of RNA interference: from functional genomics to therapeutics. (2005) Adv Genet 54: 117-42.
Miyake H, Chi and Cleave M E (2000) Antisense TRPM-2 oligodeoxynucleotides chemosensitize human androgen-independent PC-3 prostate cancer cells both in vitro and in vivo. Clin Cáncer Res, 6, 1655-63.
Peinado H, Del Carmen Iglesias-de la Cruz M, Olmeda D, Csiszar K, Fong K S, Vega S, Nieto M A, Cano A, Portillo F (2005) A molecular role for lysyl oxidase-like 2 enzyme in snail regulation and tumor progression. EMBO J. 24(19): 3446-58.
Pfaffl M W (2001) A new mathematical model for relative quantification in real-time RT-PCR. Nucleic Acids Res. 29, e45.—Pfeifer A, Verma I M (2001) Gene therapy: promises and problems. Annu Rev Genomics Hum Genet 2: 177-211.
Puerta-Ferández E et al. (2003) Ribozymes: recent advances in the development of RNA tools. FEMS Microbiology Reviews 27: 75-97.
Reynolds A, Leake D, Boese Q, Scaringe S, Marshall WS and Khvorova A (2004) Rational siRNA design for RNA interference. Nature Biotechnology 22 (3): 326-330.
Sambrook J, Fritsch E F, and Maniatis T. (1989). Molecular cloning: a laboratory manual, 2nd ed. Cold Spring Harbor Laboratory, Cold Spring Harbor, N.Y.
Tripathi M K, Misra S, Chaudhuri G (2005) Negative regulation of the expressions of cytokeratins 8 and 19 by SLUG repressor protein in human breast cells. Biochem Biophys Res Commun. 329(2): 508-15.

1-26. (canceled)

27. A pharmaceutical composition useful in the treatment of a chondrodysplasia process comprising an inhibitory compound of the Snail1 protein.

28. A pharmaceutical composition according to claim 27 further comprising pharmaceutically acceptable adjuvants and/or carriers.

29. A pharmaceutical composition according to claim 27 wherein said inhibitor compound is a nucleic acid or polynucleotide that prevents or reduces the expression of the gene that encodes the Snail1 protein.

30. A pharmaceutical composition according to claim 29 wherein said inhibitory compound is one or more of:
a) an anti-sense nucleotide sequence specific to the gene or mRNA sequence of the Snail1 protein,
b) a ribozyme specific to the mRNA of the Snail1 protein,
c) an aptamer specific to the mRNA of the Snail1 protein,
d) siRNA or shRNA specific to the mRNA of the Snail1 protein, or
e) an miRNA specific to the Snail1 protein.

31. A pharmaceutical composition according to claim 30 wherein said inhibitory compound is siRNA that binds to SEQ ID NO:17, a sequence comprising SEQ ID NO: 17 or a fragment of SEQ ID NO: 17.

32. A pharmaceutical composition according to claim 31 wherein said siRNA comprises SEQ ID NO 11, SEQ ID NO 12, SEQ ID NO 13, SEQ ID NO 14, SEQ ID NO 15 and/or SEQ ID NO 16.

33. A pharmaceutical composition according to claim 32 wherein said siRNA comprises a pair of nucleotide sequences including SEQ ID NO 11 and SEQ ID NO 12, SEQ ID NO 13 and SEQ ID NO 14 or SEQ ID NO 15 and SEQ ID NO 16.

34. A method of treating a mammal affected by a chondrodysplasia process comprising administering a therapeutically effective amount of a pharmaceutical composition of claim 27.

35. A method according to claim 34 wherein said chondrodysplasia process is achondroplasia (ACH), thanatophoric dysplasia (TD) or hypochondroplasia (HCH).

36. A method according to claim 34 wherein said inhibitory compound is siRNA that binds to SEQ ID NO:17, a sequence comprising SEQ ID NO: 17 or a fragment of SEQ ID NO: 17 and includes SEQ ID NO 11, SEQ ID NO 12, SEQ ID NO 13, SEQ ID NO 14, SEQ ID NO 15 and/or SEQ ID NO 16.

37. A method for identifying a chondrodysplasia process in a mammal comprising:
a) identifying the abberant presence of Snail1 in a biological sample of osseous origin from said mammal by
b) comparing the presence of Snail1 observed in a) to its absence in a control sample, where its presence is indicative of the existence of chondrodysplasia.

38. A method according to claim 37 wherein said identifying comprises identification of Snail1 mRNA or protein expression.

39. A method according to claim 37 wherein said identifying comprises identification of SEQ ID NO:1, SEQ ID NO:2, SEQ ID NO:3 or SEQ ID NO:4.

40. A method according to claim 37 wherein said identifying is performed using monoclonal or polyclonal antibodies of the hSnail1 protein, in situ hybridisation with a Snail1 precursor, or reverse transcriptase—polymerase chain reaction (RT-PCR) of a Snail1 precursor.

41. A method for identifying the activity of inhibitory compounds of the Snail1 protein useful in the treatment of chondrodysplasia comprising:
a) placing a biological system with an expression of Snail1 that produces chondrodysplasia in contact with a candidate compound,
b) determining a parameter that is indicative of the chondrodysplasia process, and
c) identifying a compound inhibitory of Snail1 protein activity when a reduction of said chondrodysplasia parameter is observed.

42. A method according to claim 41 wherein said biological system is a non-human transgenic animal.

43. A method according to claim 41 wherein said biological system is a non-human transgenic animal with inducible Snail1 expression.

44. A method according to claim 43 wherein said inducible Snail1 expression is constant or conditional and causes chondrodysplasia.

45. A method according to claim 42 wherein said non-human transgenic animal is the transgSnail1-ER transgenic mouse.

46. A transgSnail1-ER transgenic mouse.


SEQUENCE LISTING (17 sequences)

SEQ ID NO: 1 - DNA, 1613 bp, Mouse
  CDS (64)..(858): Mouse Snail1 coding sequence
cggagttgactaccgaccttgcgcgacccggtgaccccgactacctaggtcgctctggccaacatgccgcgctccttcctggtcaggaagccgtccgacccccgccggaagcccaactatagcgagctgcaggacgcgtgtgtggagttcaccttccagcagccctacgaccaggcccacctgctggccgccatccctccgcccgaggtcctcaaccccgccgcttcgctgcccaccctcatctgggactctctcctggtaccccaagtgcggccggttgcctgggccaccctcccgctgcgggagagccccaaggccgtagagctgacctcgctgtccgatgaggacagtggcaaaagctcccagccgcccagcccgccctcgccggcgccgtcgtccttctcgtccacctcggcctcgtccctggaggccgaggccttcatcgccttccctggcttgggccaacttcccaagcagctggccaggctctcggtggccaaggacccccagtcgcggaagatcttcaactgcaaatattgtaacaaggagtacctcagcctgggcgctctgaagatgcacatccgaagccacacgctgccttgtgtctgcacgacctgtggaaaggccttctctaggccctggctgcttcagggccacgtccgcacccacactggtgagaagccattctcctgctcccactgcaaccgtgcttttgctgaccgctccaacctgcgtgcccacctccaaacccactcggatgtgaagagataccagtgccaggcctgtgcccgaaccttctcccgcatgtccttgctccacaagcaccaagagtctggctgctccggaggccctcgctgaccctgctacctccccatcctcgctggcatcttcccggagctcaccctcctcctcactgccaggactccttccagccttggtccggggacctgtggcgtccatgtctggacctggttcctgcttggctctcttggtggcctttgccgcaggtggctgatggagtgcctttgtacccgcccagagcctcctacccctcagtattcatgaggtgtagcctctggacacagctgcttcgagccatagaactaaagccaacccactggctgggaagcttgaaccccgctcaggggaccccacttccctacctccctcaaggacccttcaggccaccttctttgaggtacaacagactatgcaatagttcccctcccccccaccccgtccagctgtaaccatgcctcagcagggtggttactggacacatgtccaggtgcccctgggcctgggcaactgtttcagcccccgcccccatttgtcctggtgacacctgtttcacagcagtttaactgtctcagaagggaccatgaataatggccatcacttgttaggggccaagtggggtgcttcagcctggccaatgtgtctcccagaactattttggggcccaacaggtggccccgggagaaagatgtttacattttaaaggtatttatattgtaagcagcattttgtatagttaatatgtacagtttattgatattcaataaaatggttaatttatatactaaaaaaaaaaaaaaaaaaaaaaaaaaaaa

SEQ ID NO: 2 - protein, 264 aa, Mouse
MPRSFLVRKPSDPRRKPNYSELQDACVEFTFQQPYDQAHLLAAIPPPEVLNPAASLPTLIWDSLLVPQVRPVAWATLPLRESPKAVELTSLSDEDSGKSSQPPSPPSPAPSSFSSTSASSLEAEAFIAFPGLGQLPKQLARLSVAKDPQSRKIFNCKYCNKEYLSLGALKMHIRSHTLPCVCTTCGKAFSRPWLLQGHVRTHTGEKPFSCSHCNRAFADRSNLRAHLQTHSDVKRYQCQACARTFSRMSLLHKHQESGCSGGPR

SEQ ID NO: 3 - DNA, 1708 bp, Homo sapiens
  CDS (71)..(865): Human Snail1 coding sequence
ggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 4 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 5 - DNA, 22 bp, Artificial
  Oligo_mGapdh_A
ctgagcaagagaggccctatcc

SEQ ID NO: 6 - DNA, 20 bp, Artificial
  Oligo_mGapdh_B
ctccctaggcccctcctgtt

SEQ ID NO: 7 - DNA, 20 bp, Artificial
  Oligo_mP21_A
aggagccaggccaagatggt

SEQ ID NO: 8 - DNA, 22 bp, Artificial
  Oligo_mP21_B
gctttgacacccacggtattca

SEQ ID NO: 9 - DNA, 22 bp, Artificial
  Oligo_mSnail1_A
ccacactggtgagaagccattc

SEQ ID NO: 10 - DNA, 22 bp, Artificial
  Oligo_mSnail1_B
tcttcacatccgagtgggtttg

SEQ ID NO: 11 - RNA, 25 nt, Artificial
  siRNA_I_A
cggaagaucuucaacugcaaauauu

SEQ ID NO: 12 - RNA, 25 nt, Artificial
  siRNA_I_Bcomplem
aauauuugcaguugaagaucuuccg

SEQ ID NO: 13 - RNA, 25 nt, Artificial
  siRNA_II_A
caaacccacucggaugugaagagau

SEQ ID NO: 14 - RNA, 25 nt, Artificial
  siRNA_II_Bcomplem
aucucuucacauccgaguggguuug

SEQ ID NO: 15 - RNA, 25 nt, Artificial
  siRNA_III_A
cagcugcuucgagccauagaacuaa

SEQ ID NO: 16 - RNA, 25 nt, Artificial
  siRNA_III_Bcomplem
uuaguucuauggcucgaagcagcug

SEQ ID NO: 17 - DNA, 19 bp, Artificial
  RNAm_snail
gatgcacatccgaagccac